SEQUENCE LISTING (88 sequences)

SEQ ID NO: 1 - protein, 122 aa, Bos taurus
  DISULFID (6)..(120)
  DISULFID (28)..(111)
  DISULFID (61)..(77)
  DISULFID (73)..(91)
EQLTKCEVFRELKDLKGYGGVSLPEWVCTTFHTSGYDTQAIVQNNDSTEYGLFQINNKIWCKDDQNPHSSNICNISCDKFLDDDLTDDIMCVKKILDKVGINYWLAHKALCSEKLDQWLCEK

SEQ ID NO: 2 - protein, 122 aa, Bos taurus; identical to SEQ ID NO: 1
  DISULFID (6)..(28)
  DISULFID (61)..(73)
  DISULFID (77)..(91)
  DISULFID (111)..(120)

SEQ ID NO: 3 - protein, 122 aa, Bos taurus; identical to SEQ ID NO: 1
  DISULFID (6)..(28)
  DISULFID (61)..(77)
  DISULFID (73)..(91)
  DISULFID (111)..(120)

SEQ ID NO: 4 - protein, 122 aa, Bos taurus; identical to SEQ ID NO: 1
  DISULFID (6)..(120)
  DISULFID (28)..(111)
  DISULFID (61)..(73)
  DISULFID (77)..(91)

SEQ ID NO: 5 - protein, 122 aa, Bos taurus; identical to SEQ ID NO: 1
  DISULFID (6)..(28)
  DISULFID (61)..(91)
  DISULFID (73)..(77)
  DISULFID (111)..(120)

SEQ ID NO: 6 - protein, 122 aa, Bos taurus; identical to SEQ ID NO: 1
  DISULFID (6)..(28)
  DISULFID (61)..(120)
  DISULFID (73)..(111)
  DISULFID (77)..(91)

SEQ ID NO: 7 - protein, 122 aa, Bos taurus; identical to SEQ ID NO: 1
  DISULFID (6)..(28)
  DISULFID (61)..(73)
  DISULFID (77)..(111)
  DISULFID (91)..(120)

SEQ ID NO: 8 - protein, 122 aa, Bos taurus; identical to SEQ ID NO: 1
  DISULFID (6)..(61)
  DISULFID (28)..(73)
  DISULFID (77)..(91)
  DISULFID (111)..(120)

SEQ ID NO: 9 - protein, 53 aa, Homo sapiens
  DISULFID (6)..(20)
  DISULFID (14)..(31)
  DISULFID (33)..(42)
NSDSECPLSHDGYCLHDGVCMYIEALDKYACNCVVGYIGERCQYRDLKWWELR

SEQ ID NO: 10 - protein, 53 aa, Homo sapiens; identical to SEQ ID NO: 9
  DISULFID (6)..(42)
  DISULFID (14)..(33)
  DISULFID (20)..(31)

SEQ ID NO: 11 - protein, 53 aa, Homo sapiens; identical to SEQ ID NO: 9
  DISULFID (6)..(14)
  DISULFID (20)..(31)
  DISULFID (33)..(42)

SEQ ID NO: 12 - protein, 53 aa, Homo sapiens; identical to SEQ ID NO: 9
  DISULFID (6)..(42)
  DISULFID (14)..(31)
  DISULFID (20)..(33)

SEQ ID NO: 13 - protein, 53 aa, Homo sapiens; identical to SEQ ID NO: 9
  DISULFID (6)..(14)
  DISULFID (20)..(33)
  DISULFID (31)..(42)

SEQ ID NO: 14 - protein, 53 aa, Homo sapiens; identical to SEQ ID NO: 9
  DISULFID (6)..(33)
  DISULFID (14)..(20)
  DISULFID (31)..(42)

SEQ ID NO: 15 - protein, 53 aa, Homo sapiens; identical to SEQ ID NO: 9
  DISULFID (6)..(42)
  DISULFID (14)..(20)
  DISULFID (31)..(33)

SEQ ID NO: 16 - protein, 53 aa, Homo sapiens; identical to SEQ ID NO: 9
  DISULFID (6)..(14)
  DISULFID (20)..(42)
  DISULFID (31)..(33)

SEQ ID NO: 17 - protein, 53 aa, Homo sapiens; identical to SEQ ID NO: 9
  DISULFID (6)..(31)
  DISULFID (14)..(20)
  DISULFID (33)..(42)

SEQ ID NO: 18 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 19 - protein, 140 aa, Homo sapiens
  DISULFID (9)..(42)
  DISULFID (69)..(89)
  DISULFID (107)..(124)
MDVFMKGLCKAKEGVVAAAEKTKQGVAEAAGKTKEGVLYVGCKTKEGVVHGVATVAEKTKEQVTNVGGCVVTGVTAVAQKTVEGAGSICAATGFVKKDQLGKNEEGCPQEGILEDMPVDPDNECYEMPSEEGYQDYEPEA

SEQ ID NO: 20 - protein, 140 aa, Homo sapiens; identical to SEQ ID NO: 19
  DISULFID (9)..(69)
  DISULFID (42)..(89)
  DISULFID (107)..(124)

SEQ ID NO: 21 - protein, 140 aa, Homo sapiens; identical to SEQ ID NO: 19
  DISULFID (9)..(89)
  DISULFID (42)..(69)
  DISULFID (107)..(124)

SEQ ID NO: 22 - protein, 140 aa, Homo sapiens; identical to SEQ ID NO: 19
  DISULFID (9)..(107)
  DISULFID (42)..(69)
  DISULFID (89)..(124)

SEQ ID NO: 23 - protein, 140 aa, Homo sapiens; identical to SEQ ID NO: 19
  DISULFID (9)..(124)
  DISULFID (42)..(69)
  DISULFID (89)..(107)

SEQ ID NO: 24 - protein, 140 aa, Homo sapiens; identical to SEQ ID NO: 19
  DISULFID (9)..(42)
  DISULFID (69)..(107)
  DISULFID (89)..(124)

SEQ ID NO: 25 - protein, 140 aa, Homo sapiens; identical to SEQ ID NO: 19
  DISULFID (9)..(69)
  DISULFID (42)..(107)
  DISULFID (89)..(124)

SEQ ID NO: 26 - protein, 140 aa, Homo sapiens; identical to SEQ ID NO: 19
  DISULFID (9)..(89)
  DISULFID (42)..(107)
  DISULFID (69)..(124)

SEQ ID NO: 27 - protein, 140 aa, Homo sapiens; identical to SEQ ID NO: 19
  DISULFID (9)..(107)
  DISULFID (42)..(89)
  DISULFID (69)..(124)

SEQ ID NO: 28 - protein, 140 aa, Homo sapiens; identical to SEQ ID NO: 19
  DISULFID (9)..(124)
  DISULFID (42)..(89)
  DISULFID (69)..(107)

SEQ ID NO: 29 - protein, 140 aa, Homo sapiens; identical to SEQ ID NO: 19
  DISULFID (9)..(42)
  DISULFID (69)..(124)
  DISULFID (89)..(107)

SEQ ID NO: 30 - protein, 140 aa, Homo sapiens; identical to SEQ ID NO: 19
  DISULFID (9)..(69)
  DISULFID (42)..(124)
  DISULFID (89)..(107)

SEQ ID NO: 31 - protein, 140 aa, Homo sapiens; identical to SEQ ID NO: 19
  DISULFID (9)..(89)
  DISULFID (42)..(124)
  DISULFID (69)..(107)

SEQ ID NO: 32 - protein, 140 aa, Homo sapiens; identical to SEQ ID NO: 19
  DISULFID (9)..(107)
  DISULFID (42)..(124)
  DISULFID (69)..(89)

SEQ ID NO: 33 - protein, 140 aa, Homo sapiens; identical to SEQ ID NO: 19
  DISULFID (9)..(124)
  DISULFID (42)..(107)
  DISULFID (69)..(89)

SEQ ID NO: 34 - protein, 210 aa, Mus musculus
SKKRPKPGGWNTGGSRYPGQGSPGGNRYPPQGGTWGQPHGGGWGQPHGGSWGQPHGGSWGQPHGGGWGQGGGTHNQWNKPSKPKTNLKHVAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSS

SEQ ID NO: 35 - protein, 210 aa, Mus musculus
  DISULFID (15)..(91)
  DISULFID (113)..(148)
  DISULFID (157)..(192)
SKKRPKPGGWNTGGCRYPGQGSPGGNRYPPQGGTWGQPHGGGWGQPHGGSWGQPHGGSWGQPHGGGWGQGGGTHNQWNKPSKPKTNLKHVCGAAAAGAVVGGLGGYMLGSAMCRPMIHFGNDWEDRYYRENMYRYPNQVYYRPVDQYCNQNNFVHDCVNITIKQHTVTTTTKGENFTETDVKMMERVVEQMCVTQYQKESQAYYDGRRSS

SEQ ID NO: 36 - protein, 210 aa, Mus musculus; identical to SEQ ID NO: 35
  DISULFID (15)..(113)
  DISULFID (91)..(148)
  DISULFID (157)..(192)

SEQ ID NO: 37 - protein, 210 aa, Mus musculus; identical to SEQ ID NO: 35
  DISULFID (15)..(148)
  DISULFID (91)..(113)
  DISULFID (157)..(192)

SEQ ID NO: 38 - protein, 210 aa, Mus musculus; identical to SEQ ID NO: 35
  DISULFID (15)..(157)
  DISULFID (91)..(113)
  DISULFID (148)..(192)

SEQ ID NO: 39 - protein, 210 aa, Mus musculus; identical to SEQ ID NO: 35
  DISULFID (15)..(192)
  DISULFID (91)..(113)
  DISULFID (148)..(157)

SEQ ID NO: 40 - protein, 210 aa, Mus musculus; identical to SEQ ID NO: 35
  DISULFID (15)..(91)
  DISULFID (113)..(157)
  DISULFID (148)..(192)

SEQ ID NO: 41 - protein, 210 aa, Mus musculus; identical to SEQ ID NO: 35
  DISULFID (15)..(113)
  DISULFID (91)..(157)
  DISULFID (148)..(192)

SEQ ID NO: 42 - protein, 210 aa, Mus musculus; identical to SEQ ID NO: 35
  DISULFID (15)..(148)
  DISULFID (91)..(157)
  DISULFID (113)..(192)

SEQ ID NO: 43 - protein, 210 aa, Mus musculus; identical to SEQ ID NO: 35
  DISULFID (15)..(157)
  DISULFID (91)..(148)
  DISULFID (113)..(192)

SEQ ID NO: 44 - protein, 210 aa, Mus musculus; identical to SEQ ID NO: 35
  DISULFID (15)..(192)
  DISULFID (91)..(148)
  DISULFID (113)..(157)

SEQ ID NO: 45 - protein, 210 aa, Mus musculus; identical to SEQ ID NO: 35
  DISULFID (15)..(91)
  DISULFID (113)..(192)
  DISULFID (148)..(157)

SEQ ID NO: 46 - protein, 210 aa, Mus musculus; identical to SEQ ID NO: 35
  DISULFID (15)..(113)
  DISULFID (91)..(192)
  DISULFID (148)..(157)

SEQ ID NO: 47 - protein, 210 aa, Mus musculus; identical to SEQ ID NO: 35
  DISULFID (15)..(148)
  DISULFID (91)..(192)
  DISULFID (113)..(157)

SEQ ID NO: 48 - protein, 210 aa, Mus musculus; identical to SEQ ID NO: 35
  DISULFID (15)..(157)
  DISULFID (91)..(192)
  DISULFID (113)..(148)

SEQ ID NO: 49 - protein, 210 aa, Mus musculus; identical to SEQ ID NO: 35
  DISULFID (15)..(192)
  DISULFID (91)..(157)
  DISULFID (113)..(148)

SEQ ID NO: 50 - protein, 42 aa, Homo sapiens [sequence omitted]

SEQ ID NO: 51 - protein, 42 aa, Homo sapiens
DCEFRHDCGYEVHHQKLVFFCEDVGCNKGCIIGLMVGGVVIC

SEQ ID NO: 52 - protein, 42 aa, Homo sapiens; identical to SEQ ID NO: 51
  DISULFID (2)..(8)
  DISULFID (21)..(26)
  DISULFID (30)..(42)

SEQ ID NO: 53 - protein, 42 aa, Homo sapiens; identical to SEQ ID NO: 51
  DISULFID (2)..(21)
  DISULFID (8)..(26)
  DISULFID (30)..(42)

SEQ ID NO: 54 - protein, 42 aa, Homo sapiens; identical to SEQ ID NO: 51
  DISULFID (2)..(26)
  DISULFID (8)..(21)
  DISULFID (30)..(42)

SEQ ID NO: 55 - protein, 42 aa, Homo sapiens; identical to SEQ ID NO: 51
  DISULFID (2)..(30)
  DISULFID (8)..(21)
  DISULFID (26)..(42)

SEQ ID NO: 56 - protein, 42 aa, Homo sapiens; identical to SEQ ID NO: 51
  DISULFID (2)..(42)
  DISULFID (8)..(21)
  DISULFID (26)..(30)

SEQ ID NO: 57 - protein, 42 aa, Homo sapiens; identical to SEQ ID NO: 51
  DISULFID (2)..(8)
  DISULFID (21)..(30)
  DISULFID (26)..(42)

SEQ ID NO: 58 - protein, 42 aa, Homo sapiens; identical to SEQ ID NO: 51
  DISULFID (2)..(21)
  DISULFID (8)..(30)
  DISULFID (26)..(42)

SEQ ID NO: 59 - protein, 42 aa, Homo sapiens; identical to SEQ ID NO: 51
  DISULFID (2)..(26)
  DISULFID (8)..(30)
  DISULFID (21)..(42)

SEQ ID NO: 60 - protein, 42 aa, Homo sapiens; identical to SEQ ID NO: 51
  DISULFID (2)..(30)
  DISULFID (8)..(26)
  DISULFID (21)..(42)

SEQ ID NO: 61 - protein, 42 aa, Homo sapiens; identical to SEQ ID NO: 51
  DISULFID (2)..(42)
  DISULFID (8)..(26)
  DISULFID (21)..(30)

SEQ ID NO: 62 - protein, 42 aa, Homo sapiens; identical to SEQ ID NO: 51
  DISULFID (2)..(8)
  DISULFID (21)..(42)
  DISULFID (26)..(30)

SEQ ID NO: 63 - protein, 42 aa, Homo sapiens; identical to SEQ ID NO: 51
  DISULFID (2)..(21)
  DISULFID (8)..(42)
  DISULFID (26)..(30)

SEQ ID NO: 64 - protein, 42 aa, Homo sapiens; identical to SEQ ID NO: 51
  DISULFID (2)..(26)
  DISULFID (8)..(42)
  DISULFID (21)..(30)

SEQ ID NO: 65 - protein, 42 aa, Homo sapiens; identical to SEQ ID NO: 51
  DISULFID (2)..(30)
  DISULFID (8)..(42)
  DISULFID (21)..(26)

SEQ ID NO: 66 - protein, 42 aa, Homo sapiens; identical to SEQ ID NO: 51
  DISULFID (2)..(42)
  DISULFID (8)..(30)
  DISULFID (21)..(26)

SEQ ID NO: 67 - protein, 42 aa, Homo sapiens [sequence omitted]

SEQ ID NO: 68 - protein, 42 aa, Homo sapiens
DCEFRHDCGYEVHHQKLVFFCEDVGSNKGCIIGLMVGGVVIC

SEQ ID NO: 69 - protein, 42 aa, Homo sapiens; identical to SEQ ID NO: 68
  DISULFID (2)..(8)
  DISULFID (21)..(30)
  MOD_RES (42): Cys-SH

SEQ ID NO: 70 - protein, 42 aa, Homo sapiens; identical to SEQ ID NO: 68
  DISULFID (2)..(8)
  DISULFID (21)..(42)
  MOD_RES (30): Cys-SH

SEQ ID NO: 71 - protein, 42 aa, Homo sapiens; identical to SEQ ID NO: 68
  DISULFID (2)..(8)
  MOD_RES (21): Cys-SH

SEQ ID NO: 72 - protein, 42 aa, Homo sapiens; identical to SEQ ID NO: 68
  DISULFID (2)..(21)
  MOD_RES (8): Cys-SH

SEQ ID NO: 73 - protein, 42 aa, Homo sapiens; identical to SEQ ID NO: 68
  MOD_RES (2): Cys-SH

SEQ ID NO: 74 - protein, 42 aa, Homo sapiens; identical to SEQ ID NO: 68
  DISULFID (2)..(21)
  DISULFID (8)..(30)
  MOD_RES (42): Cys-SH

SEQ ID NO: 75 - protein, 42 aa, Homo sapiens; identical to SEQ ID NO: 68
  DISULFID (2)..(21)
  DISULFID (8)..(42)
  MOD_RES (30): Cys-SH

SEQ ID NO: 76 - protein, 42 aa, Homo sapiens; identical to SEQ ID NO: 68
  DISULFID (2)..(30)
  DISULFID (8)..(42)
  MOD_RES (21): Cys-SH

SEQ ID NO: 77 - protein, 42 aa, Homo sapiens; identical to SEQ ID NO: 68
  DISULFID (2)..(30)
  MOD_RES (8): Cys-SH

SEQ ID NO: 78 - protein, 42 aa, Homo sapiens; identical to SEQ ID NO: 68
  MOD_RES (2): Cys-SH

SEQ ID NO: 79 - protein, 42 aa, Homo sapiens; identical to SEQ ID NO: 68
  DISULFID (2)..(30)
  DISULFID (8)..(21)
  MOD_RES (42): Cys-SH

SEQ ID NO: 80 - protein, 42 aa, Homo sapiens; identical to SEQ ID NO: 68
  DISULFID (2)..(42)
  DISULFID (8)..(21)
  MOD_RES (30): Cys-SH

SEQ ID NO: 81 - protein, 42 aa, Homo sapiens; identical to SEQ ID NO: 68
  DISULFID (2)..(42)
  DISULFID (8)..(30)
  MOD_RES (21): Cys-SH

SEQ ID NO: 82 - protein, 42 aa, Homo sapiens; identical to SEQ ID NO: 68
  DISULFID (2)..(42)
  MOD_RES (8): Cys-SH

SEQ ID NO: 83 - protein, 42 aa, Homo sapiens; identical to SEQ ID NO: 68
  MOD_RES (2): Cys-SH

SEQ ID NO: 84 - protein, 42 aa, Homo sapiens [sequence omitted]

SEQ ID NO: 85 - protein, 42 aa, Homo sapiens
DCEFRHDSGYEVHHQKLVFFCEDVGSNKGAIIGLMVGGVVIC

SEQ ID NO: 86 - protein, 42 aa, Homo sapiens; identical to SEQ ID NO: 85
  DISULFID (2)..(21)
  MOD_RES (42): Cys-SH

SEQ ID NO: 87 - protein, 42 aa, Homo sapiens; identical to SEQ ID NO: 85
  DISULFID (2)..(42)
  MOD_RES (21): Cys-SH

SEQ ID NO: 88 - protein, 42 aa, Homo sapiens; identical to SEQ ID NO: 85
  MOD_RES (2): Cys-SH

(19) United States
(12) Patent Application Publication
(10) Pub. No.: US 2006/0018918 A1
(43) Pub. Date: Jan. 26, 2006

(54) Production of stabilized conformational isomers of disease associated proteins

(75) Inventor: Rowen J.Y. Chang, Houston, TX (US)

Correspondence Address: CHALKER FLORES, LLP, 2711 LBJ FRWY, Suite 1036, DALLAS, TX, 75234, US

(73) Assignee: Board Of Regents, The University Of Texas System, Austin, TX (US)

(21) Appl. No.: 11/177,509

(22) Filed: Jul. 8, 2005

Related U.S. Application Data
(63) Continuation-in-part of application No. 10/210,862, filed on Aug. 1, 2002.
(63) Continuation-in-part of application No. 10/025,976, filed on Dec. 19, 2001, now Pat. No. 6,900,036.
(60) Provisional application No. 60/309,543, filed on Aug. 1, 2001.
(60) Provisional application No. 60/258,576, filed on Dec. 27, 2000.

(51) Int. Cl. G01N 33/53; A61K 39/00; C40B 40/10

(52) U.S. Cl. 424/185.1; 435/7.1; 530/350

(57) ABSTRACT

The present invention includes compositions and methods for the development, isolation and characterization of vaccine that include one or more isolated and purified non-native, stabilized conformational protein isomer antigens.

This application is a Continuation-in-Part of, and claims priority to, U.S. patent application Ser. Nos. 10/210,862 filed Aug. 1, 2002, Ser. No. 10/025,976 filed Dec. 19, 2001, now U.S. Pat. No. 6,900,036, and Ser. No. 60/258,576 filed Dec. 27, 2000, the entire contents of each are incorporated herein by reference. Without limiting the scope of the invention, its background is described in connection with protein isomers.

### TECHNICAL FIELD OF THE INVENTION

The present invention relates in general to the field of protein isomers, and more particularly, to generating selective populations of immunogenic, non-native, stable protein isomers by optimizing the conditions for their formation.

### BACKGROUND OF THE INVENTION

A protein can potentially assume an exceedingly large number of conformations. Under physiological conditions, a protein usually folds “properly” and adopts the native structure with a well-defined, three-dimensional conformation. In contrast to a properly folded protein, a denatured protein includes a collection of conformational isomers that exist in a state of equilibrium, with a wide variety of three-dimensional conformations.

Conventional approaches used to study protein folding involve unfolding proteins in the presence of a strong denaturant (e.g., 8M Urea or 6M Guanidinium Chloride (GdmCl), Guanidinium isothiocyanate (GdmSCN), phenol and other organic solvents), extreme pH or high temperature. Following the removal of the denaturant, reduction of pH, or reduction of temperature, the denatured proteins refold spontaneously to form, e.g., the native structure. The refolding pathway of the protein can be monitored by the restoration of one or more physicochemical readouts that distinguish the native and non-native folding of a protein. Commonly used physicochemical readouts are spectra of fluorescence, circular dichroism, infrared, ultraviolet light and nuclear magnetic resonance (NMR) coupled with amide proton exchange. Unfortunately, in most cases these methods do not permit the isolation, characterization and purification of large amounts of a specific folding intermediate.

Another method used to study protein folding is the oxidative folding of disulfide bonds in proteins that have at least two sulfhydryl groups. Proteins are reduced and denatured in the presence of a reducing agent, such as dithiothreitol (DTT), and a denaturant, such as 6M GdmCl. After exclusion of the reductant and denaturant, the reduced and denatured protein is allowed to refold in the presence of redox buffer. The refolding pathway is then tracked by the mechanisms of formation of the native disulfide bonds. For example, a protein that contains three disulfide bonds can potentially assume 75 different disulfide isomers (15 isomer species having one disulfide bond, 45 having two disulfide bonds, and 15 having three disulfide bonds). The disulfide folding pathway is characterized by the heterogeneity and structures of the disulfide isomers that accumulate in the process of oxidative folding that leads to formation of the native structure. However, without chemical modification, the method of oxidative folding does not generate stable isomers.

### SUMMARY OF THE INVENTION

The present invention is based on the recognition that diverse, non-native protein isomers may be used for the treatment of a wide variety of diseases, e.g., infectious diseases. The present invention includes the development, isolation and characterization of disease-associated non-native protein isomers. It is demonstrated herein that non-native protein isomers can be designed, produced and isolated that demonstrate immunogenicity. The denatured protein isomers of the present invention were found to exhibit more potent immunogenicity than the native protein.

Yet another aspect of the present invention is the development of a method for making these unique non-native protein isomers by determining and using a combination of denaturant with an optimized concentration of thiol initiator for converting the native protein to a mixture of fully oxidized scrambled isomers. These selective populations of non-native, disulfide isomers were found to have particular uses in vaccine development due to the enhanced immunogenicity.

The method, strategy and compositions of the present invention are useful for the production of selected populations of stabilized isomers of proteins as vaccines. The method permits the production of increased number of stabilized isomers of proteins by insertion/addition of cysteines. One example of the production of stabilized isomers is the use of human epidermal growth factor (EGF) for the intervention and treatment of EGF associated diseases. Another example of the production of stabilized isomers of amyloid β-protein (Aβ) for the intervention and treatment of Alzheimer's disease. Further examples include the production of: stabilized isomers of α-synuclein for the intervention and treatment of Parkinson's disease and related diseases; and stabilized isomers of prion protein for the intervention and treatment of Prion diseases, including, e.g., mad cow diseases and Creutzfeldt-Jacob disease.

The present invention also includes a customizable method for development of vaccines using diverse and stabilized conformational isomers of relevant proteins. For example, the present may be used for rapid vaccine developed by providing multiple isomer for a single diseases which include, but not limited to, the above mentioned diseases. For example, a stabilized conformational isomer of fragments of gp120 can be prepared as candidates of HIV vaccine by this approach. Also, stabilized isomers of VEGF can be prepared as candidates of therapeutic agent to elicit and boost the immune response to contain the function of VEGF. Another example includes stabilized isomers of cell-surface domains of receptors as therapeutic agents that elicit immune response to block the respective receptor and diminish the receptor mediated functions.

The compositions and methods of the present invention also relate to the production and application of stabilized protein isomers, in purified form or in mixture form, as candidates of vaccine development for prevention and treatment of human diseases that are patient specific. For example, multiple isomers, or pools of isomers may be spotted, e.g., subcutaneously, to determine the best immune response. Following the initial inoculation, the best immunogenic isomer or pool of isomers may be used for continued inoculation. The stronger immune response may not always be the best immune response for continued inoculation, e.g., some isomers may tend to elicit an immune response of a particular antibody isotype, when another may be preferred.

The present inventors recognized that non-native, conformational isomers represent a vast resource of biological molecules that have, thus far, remained untapped. The major obstacle to using the untapped potential of conformation isomers is the inherent difficulty in the isolation and characterization of pure conformational isomers, not only because of the excessive large number that may exist but also because of their instability and rapid inter-conversion.

The present invention includes one or more vaccine that includes one or more isolated and purified non-native, stabilized conformational protein isomer antigens. The protein isomer will generally include three or more cysteines and may be, e.g., a wild-type protein that has been modified to comprise three or more cysteines. The residues for modification into a sulfydryl side-group may be selected a group that includes, e.g., Alanine, Serine and Threonine and are modified to one or more cysteines. The vaccine may include one or more pools of protein isomers. Often, the vaccine may also be provided with one or more adjuvants. The vaccine may be further adapted for intramuscular, intravenous, subcutaneous, pulmonary, oral, ocular, topical, sublingual, intraperitoneal, intraosseal, rectal, vaginal or intranasal injection.

Non-limiting examples of proteins for use as vaccines of the present invention include prion protein, α-synuclein, amyloid β-protein, CD4 or gp120. More particularly, the isomer may be associated with a conformational disease selected from the group consisting of prion-associated diseases, mad cow disease, scrapie, Creutzfeldt-Jacob disease, familial insomnia, Alzheimer disease, Parkinson disease, α1-antitrypsin deficiency and cystic fibrosis. Alternatively, the protein selected for the production of non-native isomers includes those from pathogenic organisms, proteins associated with auto-immune diseases, cancers, auto-inflammatory diseases, allergies, anaphylaxis and the like. The vaccine of the presenting invention may further include one or more protein isomers that form protein aggregates.

Another embodiment of the present invention includes a non-native, protein isomer library that includes one or more non-native, stabilized conformational protein isomers antigens. The library may even include one or more pools of pools of non-native, stabilized protein isomers.

The invention also includes a method for producing non-native and stabilized protein isomer antigens by contacting a protein antigen that includes three or more sulfydryl side-groups in the presence of varying denaturing conditions against one or more concentrations of a thiol agent. The thiol agent may be selected from 2-mercaptoethanol, reduced glutathione, cysteine, dithiothreitol, and thiol-containing chemicals. Non-limiting examples of denaturing conditions include one or more concentrations of Urea, guanidinium chloride, guanidinium isothiocyanate, organic solvents, and mixture or combinations thereof. One or more denaturing conditions may be selected from the group consisting of urea, guanidine hydrochloride, guanidinium isothiocyanate, organic solvents, elevated temperature, extreme pH, surfactants and detergents, and mechanical forces such as shaking, shearing, ultrasound, radiation and pressure. For example, the protein may be exposed to temperatures ranging from between about 0 and 90 degrees centigrade.

In another embodiment, the method for producing a non-native, stabilized protein isomer antigen includes contacting a protein antigen with two or more sulfydryl groups in the presence of varying denaturing conditions against one or more concentrations of a thiol agent. The two or more sulfydryl groups may be native to the protein or may be added through recombinant methods well-known in the art. In one example, the method for isolating a stable, non-native conformational protein isomer may include the steps of: contacting a protein in a titration array in one or more wells with one or more concentrations of a protein denaturing agent and one or more concentrations of a reducing agent; and vaccinating an animal with the one or more a stable, non-native conformational protein isomers formed in the one or more cells. The one or more stable, non-native conformational protein isomers from different wells are compared for immunogenicity. Alternatively, the one or more stable, non-native conformational protein isomers from different wells are compared for cross-reactivity. Examples of reducing agents include, e.g., 2-mercaptoethanol, reduced glutathione, cysteine, dithiothreitol, and thiol-containing chemicals. Various protein denaturing agents and conditions may include: one or more concentrations of Urea, guanidinium chloride, guanidinium isothiocyanate, organic solvents, and mixture or combinations thereof, which may be titrated to obtain multiple isomers and conformations. These isomers may be further purified and isolated using techniques well known in the art, e.g., precipitation, affinity chromatography, high performance liquid chromatography, fast protein liquid chromatography, two and three-dimensional gel electrophoresis, MALDI, MALDI-TOF, and combinations thereof. For example, the vaccine may include one or more non-native, immunogenic proteins from an Aβ protein comprising six cysteine residues at positions 2, 8, 21, 26, 30 and 42. Alternatively, the vaccine may include one or more non-native, immunogenic proteins of a prion protein comprising four or more cysteine residues at positions 36, 113, 135 and 170.

### BRIEF DESCRIPTION OF THE DRAWINGS

For a more complete understanding of the features and advantages of the present invention, reference is now made to the detailed description of the invention along with the accompanying figures and in which:

FIG. 1 is a diagram that shows the basic technique of disulfide scrambling;

FIG. 2 is a map which shows that α-Lactalbumin contains 122 amino acids and 4 disulfide bonds (top) and can potentially adopt 10⁴ different disulfide isomers (bottom);

FIGS. 3A to 3F are graphs that show selected population of isomers of α-lactalbumin (shown here by their HPLC profiles) can be prepared by denaturing the native protein under varied conditions: (A) 65° C., 5 min.; (B) Acetonitrile (40% by volume), CaCl₂ (5 mM), 2 h.; (C) GdmCl (1.75M), 24 h.; (D) GdmSCN (0.75M), 24 h.; (E) GdmCl (8M), 24 h.; (F) GdmSCN (6M), 24 h;

FIG. 4 are maps that show the disulfide structures of the native and 7 denatured isomers of α-Lactalbumin;

FIG. 5 includes four graphs that show the basic thermal denaturation of EGF;

FIG. 6 are maps that show the disulfide structures of the native EGF and eight isomers of scrambled EGF;

FIG. 7 are graphs of thermal denaturation for two EGF(mutants): N-EGF(S2C) and N-EGF(S4C);

FIG. 8 is a sequence and maps that show the effect of Ala/Ser→Cys mutations of α-synuclein sequence for the preparation of α-synuclein(6C) and the 15 potential isomers of α-synuclein(3SS) that may be produced from α-synuclein(6C) via disulfide oxidation and scrambling;

FIG. 9 are graphs that show the HPLC separation of isomers of α-synuclein(3SS)(left), and a demonstration that isomers of α-synuclein(3SS) exist in equilibrium under non-denaturing conditions (right);

FIG. 10 is a sequence and maps that show the Ala/Ser→Cys mutations of mPrP sequence for the preparation of mPrP(6C) and the 15 potential isomers of mPrP(3SS) that may be generated from disulfide oxidation and scrambling of mPrP(6C);

FIG. 11 are graphs that shows the oxidative folding of reduced mPrP(6C)(left); and the production of isomers of prion protein via disulfide scrambling of N-mPrP(3SS)(right); and

FIG. 12 are sequences and maps that show the Ala/Ser→Cys replacements of Aβ42 for the synthesis of Aβ42(6C), Aβ42(5C) and Aβ42(3C), and potential isomers that may be generated.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention provides a new approach to vaccine development by allowing the rapid generation of multiple isomers of a single non-native protein. The rapid development of many multiples of antigenic determinants is accomplished for any single protein (or even groups of proteins or fragments thereof) by denaturing and re-naturing a protein under different conditions to create one or more pools of potential antigens. Unlike techniques such as “expressed library immunization” that identify a wide array of many, many proteins, the present invention provides many potential antigenic epitopes for both B-cell and T-cell activation, and immunogenic combinations thereof, for a single protein or peptide. The immunization pool of antigens of the invention eliminates the need for shuffled antigen libraries to identify novel antigen isomers of the same protein. That pool of antigens that is most protective is selected from these pools by in vivo challenge models. The methods of the invention also enable the identification of individual non-native protein isomers that are antigenic in an individual patient, providing thereby a potential customized, patient-specific antigen.

The effectiveness of an antigen in inducing an immune response against a particular antigen can depend upon several factors, many of which are not well understood. Most previously available methods for increasing the effectiveness of antigens are dependent upon understanding the molecular basis for these factors. However, immunization with a pool of non-native protein isomers according to the methods of the invention are effective even where the molecular bases are unknown. The methods of the invention do not rely upon a priori assumptions or calculations of potential antigenicity because dozens, hundreds or even thousands of potential variants may be tested alone or as pools of antigens. The techniques taught by the present invention may be used to generate antigenic pools for pathogens and auto-immune disorders. For example, specific proteins from viruses, bacteria, fungi, helminths and the like may be generated. Alternatively, the immune response may be redirected (e.g., from Th1 to Th2) by immunizing with an antigenic, non-native isomer that re-directs the immune response, e.g., in response to auto-immune diseases, auto-inflammatory disease, allergies, anaphylactic shock, cancers, contraception or other host proteins that cause a condition or disease.

As used herein, the term “disulfide” is used to define the bond formed between a sulfhydryl group of, e.g., one cysteine amino acid reside, and a sulfhydryl group of, e.g., a second cysteine amino acid residue. The two cysteine residues bound together by a disulfide bond are referred to as a “cystine” residue. As used herein, the term “disulfide” is equivalent to and interchangeable with disulfide bond, disulfide bridge, disulfide crosslink, and all other applicable terms and phrases known and used by one of skill in the art.

As used herein, the term “conformation” is used to define the spatial arrangement of amino acid residues of a protein/peptide. The term “conformation” is equivalent to and interchangeable with tertiary structure, three-dimensional structure, spatial arrangement, and all other applicable terms and phrases known and used by one of skill in the art. As used herein the term “conformational disease” is used defined a disease that results from a non-native conformation of a protein, e.g., sickle cell anemia is a “conformational disease” because the disease is caused by a change to the conformation of hemoglobin.

As used herein, the term “native disulfide” is used to defined a disulfide bond in a “native” or “wild-type” protein resulting from the pairing between a sulfhydryl group on, e.g., one cysteine residue with a sulfhydryl group on, e.g., a second cysteine residue, wherein such pairing is “native” or “wild-type” pairing of sulfhydryl groups (i.e., native pairing of cysteine amino acid residues) and exists in the native conformation of a protein.

As used herein, the term “non-native disulfide” is used to define a disulfide bond in a protein resulting from the pairing between a sulfhydryl group on, e.g., one cysteine residue with a sulfhydryl group on, e.g., a second cysteine residue wherein such pairing is non-native pairing of sulfhydryl groups each or both resulting from the change or addition of, e.g., a cysteine at a location not associated with the “native” or “wild-type” amino acid sequence of the peptide or protein (i.e., non-native pairing of cysteine amino acid residues) and does not exist in the native conformation of a protein. Thus, a non-native disulfide bond may exist in the scrambled disulfide isomers of the invention between a native and another native sulfhydryl group (but not between normally associated native or wild-type bonding residues), a native and a non-native sulfhydryl group or between two non-native sulfhydryl groups. These proteins that have non-native disulfide bonds may be further isolated and purified alone or in pools for use with the present invention.

As used herein, “scrambled disulfide isomer” and “scrambled isomer” are used to define a conformational isomer of a native protein in which the scrambled isomer has at least one non-native disulfide, at least two non-native disulfides or more and the isomer has a non-native protein conformation. Combinations of scrambled isomers may also include native disulfides in addition to the at least one non-native disulfide. Each different species of scrambled disulfide isomer of the invention has a unique species-specific protein conformation and each of the species will differ from one another by at least one non-native disulfide. That is, each of the species of protein isomers includes at least one unique pairing of cysteine residues wherein the pairing is not found in the other species or in the native protein. The isomers of the invention may differ from one another and from the native protein by at least one, two or more non-native disulfide, e.g., at least two species-specific pairings of cysteine residues.

As used herein, the word “patient” may include any and all organisms capable of developing a conformation disease in which the disease is associated with conformational change of a disulfide isomer. For example, the patient of may be a vertebrate, a mammal, or even a human.

A protein can potentially assume an exceeding large number of conformations. Under physiological conditions, a protein usually folds properly and adopts the native structure with a well defined three dimensional conformation (1). Unlike the native protein, a denatured protein includes a collection of conformational isomers that exist in a state of equilibrium (2,3). Conformational isomers of denatured proteins are rich in number and varied in shape. They represent a vast resource of biological molecules that have remained untapped for their potential applications in the prevention, diagnosis and treatment of human diseases. Isomers of denatured protein are potential resource for vaccine development that has yet to be systematically exploited. Isomers of denatured proteins have been shown to involve in the development of numerous neurodegenerative diseases. They are potential targets for disease diagnosis and intervention. Isomers of denatured proteins are also potential candidates to be developed as antagonists.

The major obstacle in using this untapped resource is that isomers of denatured proteins are notoriously heterogeneous and unstable. Denatured proteins are inherently difficult to isolate and characterize for the following reasons: (a) separation of denatured isomers is a major hurdle; (b) identification of non-native conformational isomers is elusive even if they can be chromatographically fractionated from the native species; and (c) the limit of detection of the techniques currently available is another key barrier to the identification of non-native isomers. Non-native proteins include highly heterogeneous isomers (2,3), which usually exist in rapid equilibrium with the native structure and elute collectively by most chromatographic techniques. Most non-native isomers are devoid of biological function and not recognizable by antibodies directed against the native structure. Also, the majority of proteins that do fold into multiple structures, the non-native species may constitute less than 1% of the total protein under physiological conditions. This 1% of non-native proteins further includes heterogeneous isomers. Ultimately, a single isomer may represent far less than 0.1% of the total protein analyzed. It is challenging for most chromatographic systems to pick up fractions that are less than 0.1-0.2% of the predominant one, unless their identity is known and the sample is heavily overloaded.

In order to exploit applications of diverse isomers of denatured proteins, methods that able to generate significant concentration of desired isomers in purified and stable form are required. This invention is related to: (a) the design, production and isolation of stabilized conformational isomers of disease-associated proteins; and (b) the potential therapeutic application of stabilized isomers of disease-associated proteins. These proteins include, but are not limited to, human epidermal growth factor (EGF), amyloid β-protein (Aβ) for Alzheimer's disease, α-synuclein for Parkinson disease and prion protein for prion diseases.

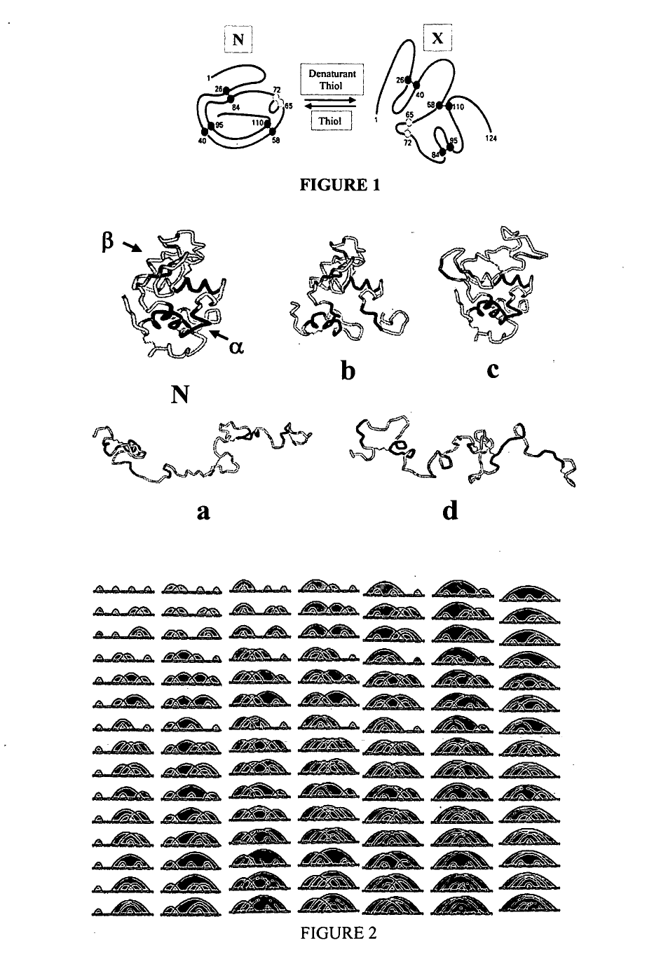

Preparation of stabilized conformational isomers of disulfide proteins. Stabilized conformational isomers of denatured proteins can be produced by the technique of disulfide scrambling (4, 5). In the presence of denaturant and a thiol initiator, a disulfide containing protein denatures by shuffling its native disulfide bonds and converts to a mixture of scrambled disulfide isomers that are trapped by non-native disulfide bonds (FIG. 1). This chemical process of denaturation can be halted at any time point by acidification or by removal of the thiol initiator. For proteins that contains 3, 4, 5, 6 and 7 disulfide bonds, respectively, there exist 15, 105, 945, 10395, and 135135, possible disulfide isomers. An example of isomers generated from a 4-disulfide protein is shown in FIG. 2. Scrambled disulfide isomers are intra-crosslinked by different sizes of disulfide loops. They are not inter-convertible in the absence of thiol catalyst or at acidic pH. Because of their stability, diverse conformations and varying physicochemical properties, scrambled isomers can be separated and purified by liquid chromatography, and structurally characterized (4, 5). Furthermore, the composition of denatured isomers is dependent upon the denaturing condition. This allows preparation from a selected protein of a desired number and composition of conformational isomers with defined structures.

The technique of disulfide scrambling is applicable in principle to the proteins that comprise at least 3 Cys. In cases of proteins containing less than 3 Cys, isomers of respective proteins can be generated by either insertion of extra Cys or replacement of existing amino acids by Cys in their amino acid sequences. Additional diversity of conformational isomers can be produced by shifting the sequence positions of Cys residues.

FIG. 1. The technique of disulfide scrambling. In the presence of denaturant and thiol catalyst, such as 2-mercaptoethanol, a disulfide containing protein shuffles its native disulfide bonds and converts to a mixture of scrambled isomers. A protein that contains four disulfides, as shown here, can adopt 104 possible scrambled isomers (see FIG. 2).

### EXAMPLE I

Production of stabilized isomers of α-lactalbumin. α-Lactalbumin is the regulatory subunit of lactose synthetase. It is one of the most extensively investigated models for understanding the protein stability, folding and unfolding (6,7). α-Lactalbumin contains 122 amino acids, four disulfide bonds. Denaturation of native α-lactalbumin can potentially generate 104 scrambled isomers (FIG. 2).

This example demonstrates that diverse populations of stabilized isomers of a protein can be produced using the technique of disulfide scrambling. Specifically, selected populations of denatured isomers of α-lactalbumin were produced by using selected denaturing conditions (5).

The native protein (0.1-20 mg/ml) was dissolved in the alkaline buffer (20-200 mM, pH 7.0-8.5) containing 0.05-0.4 mM of thiol catalyst (e.g., 2-mercaptoethanol) and selected conditions of denaturants (urea, GdmCl, GdmSCN, organic solvents, elevated temperature etc.). The reaction was allowed to reach equilibrium and was typically performed at 23° C. for 24 hours. Thiol agents other than 2-mercaptoethanol can also be used as thiol catalyst. The denatured sample was subsequently acidified with an equal volume of 4% trifluoroacetic acid and stored at −20° C. For large scale production, denaturant and thiol agent were removed by gel filtration (e.g., PD-10 or NAP-5 columns from Pharmacia AG), eluted with 1% trifluoroacetic acid. Denatured scrambled isomers are totally stable at −20° C. for at least one year. They can be typically fractionated and isolated by reversed phase HPLC.

The present invention provides, for the first time, a method for optimizing the protein isomer by a combination of denaturant with an optimized concentration of thiol initiator for converting the native protein to a mixture of fully oxidized scrambled isomers. Using various models of disulfide containing proteins it was shown that an optimized concentration of thiol initiator needs to be established case by case. For 2-mercaptoethanol, it ranges from 0.05 mM to 0.4 mM. At higher concentration of 2-mercaptoethanol (>0.4 mM), denatured scrambled isomers will become partially reduced. At lower concentrations of 2-mercaptoethanol, the efficiency for the-disulfide scrambling may be diminished.

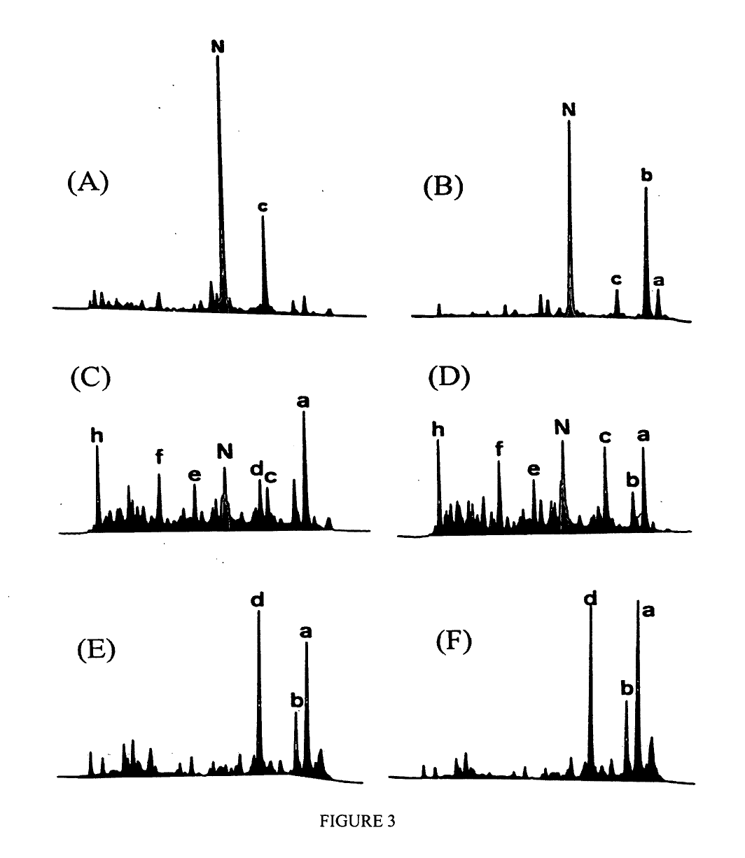

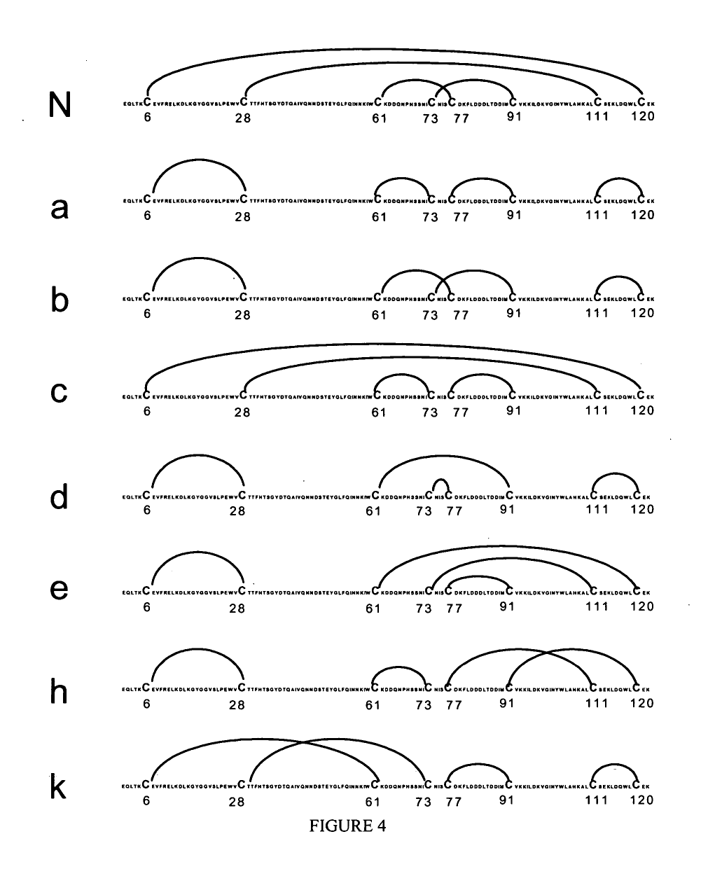

Isomers. Using the technique of disulfide scrambling, denatured α-lactalbumin was shown to consist of at least 50 fractions of scrambled isomers (FIG. 3). Among them, the disulfide structures of seven major scrambled isomers (marked from a to h) have been determined (FIG. 4). Two of them (a and d) are extensively denatured species, and two (b and c) are partially denatured species that include partly structured and partly unstructured domains. Production of favored isomers can be achieved by choosing different denaturing conditions. Examples: (1) To produce high concentration of isomer c, apply thermal denaturation (65° C.) (FIG. 3A). (2) To generate high concentration of isomer b, apply organic solvent as denaturant (30-40% acetonitrile) (FIG. 3B); (3) To generate the isomers of α-lactalbumin with a maximized heterogeneity, use mild concentration of GdmCl (1.25 M) (FIG. 3C), or GdmSCN (0.75 M) (FIG. 3D); (4) To produce high concentrations of isomers a and d, use high concentration of GdmCl (8 M) (FIG. 3E) or GdmSCN (6 M) (FIG. 3F). FIG. 2 shows that α-lactalbumin contains 122 amino acids and 4 disulfide bonds (top). α-lactalbumin can potentially adopt 10⁴ different disulfide isomers (bottom drawings). About 50 fractions of scrambled isomers were generated by chemical denaturation of α-lactalbumin.

FIG. 3. Selected population of isomers of α-lactalbumin (shown here by their HPLC profiles) can be prepared by denaturing the native protein under varied conditions. (A) 65° C., 5 min. (B) Acetonitrile (40% by volume), CaCl2 (5 mM), 2 h. (C) GdmCl (1.75M), 24 h. (D) GdmSCN (0.75M), 24 h. (E) GdmCl (8M), 24 h. (F) GdmSCN (6M), 24 h. All reactions were performed in the Tris-HCl buffer (0.1 M, pH 8.4) containing β-mercaptoethanol (0.2 mM). Aside from thermal denaturation, all reactions were carried out at 23° C. “N” (blue color) indicates the native species. Samples were analyzed by HPLC using the following conditions. Solvent A for HPLC was water with 0.1% trifluoroacetic acid. Solvent B was acetonitrile/water (9:1, by volume) containing 0.086% trifluoroacetic acid. The gradient was 22% B to 37% B in 15 min, 37% B to 56% B from 15to 45 min. The flow rate was 0.5 ml/min. Column was Zorbax 300SB C-18 for peptides and proteins, 4.6 mm, 5 mm. The predominant isomers (a to h) of denatured α-lactalbumin are marked.

FIG. 4. The disulfide structures of the native and 7 denatured isomers of α-Lactalbumin. The structures were derived from analysis of their thermolytic peptides by both Edman sequencing and MALDI mass spectrometry.

### EXAMPLE II

Production of stabilized isomers of human Epidermal Growth Factor (EGF). Human epidermal growth factor (EGF) is a 6 kd polypeptide that stimulates the growth of epidermal and epithelial cells by binding to the EGF receptor (8). This 53-amino acid growth factor adopts a well defined 3-D structure and contains three disulfide bonds with the pairing pattern of (1-3,2-4,5-6)(Cys⁶-Cys²⁰, Cys¹⁴-Cys³¹, Cys³³-Cys⁴²)(9). Denatured EGF therefore can potentially adopt 14 different scrambled isomers. EGF-like domain plays a wide ranging biological role and has been found in a large number of functional unrelated proteins. It occurs in more than 300 different sequences (10, 11).

The objective is to produce diverse and stabilized conformational isomers of human EGF as potential compounds for the intervention and treatment of EGF associated diseases. Specifically, it is expected that some of these EGF isomers will function as potent antigens that elicit production of antibodies capable of neutralizing native EGF or reducing the concentration of circulating EGF.

The native EGF (0.1-20 mg/ml) was dissolved in the alkaline buffer (20-200 mM, pH 7.0-8.5) with 0.05-0.4 mM of thiol catalyst (e.g., 2-mercaptoethanol) and selected conditions of denaturants (urea, GdmCl, GdmSCN, organic solvents, elevated temperature etc.). The reaction was allowed to reach equilibrium and was typically performed at 23° C. for 24 hours. Thiol agents other than 2-mercaptoethanol may also be used as thiol catalyst. The denatured sample was subsequently acidified with an equal volume of 4% trifluoroacetic acid and stored at −20° C. For large scale production, denaturant and thiol agent were removed by gel filtration (e.g., PD-10 or NAP-5 columns from Pharmacia AG), eluted with 1% trifluoroacetic acid. Denatured scrambled isomers are totally stable at −20° C. for at least one year. They can be typically fractionated and isolated by reversed phase HPLC.

To increase the number of EGF isomers, EGF variants with an additional Cys can be prepared by site-directed mutagenesis. For instance, EGF mutants with Ser²→Cys, Ser⁴→Cys or Ser⁹→Cys replacements can potentially generate 104 EGF isomers.

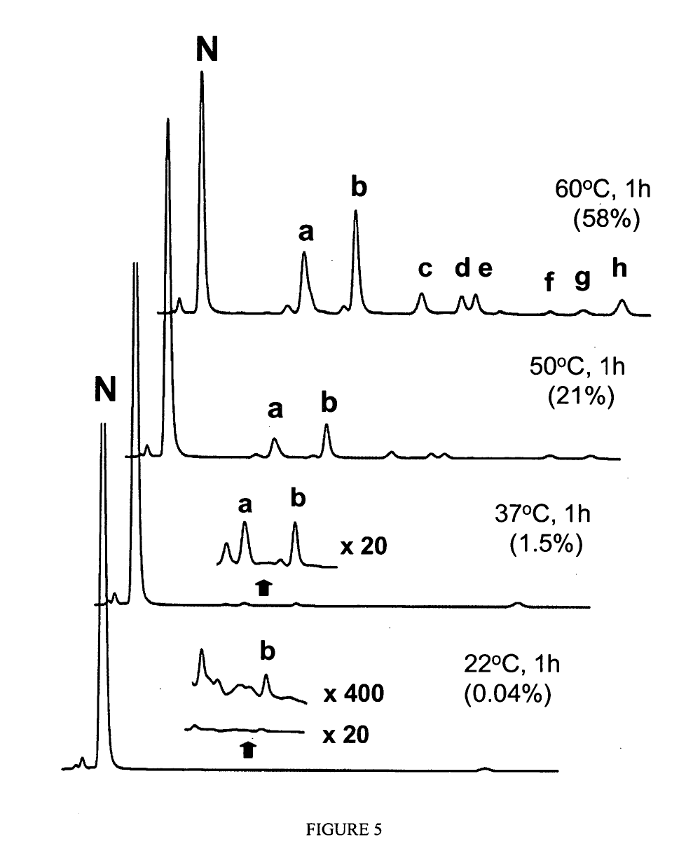

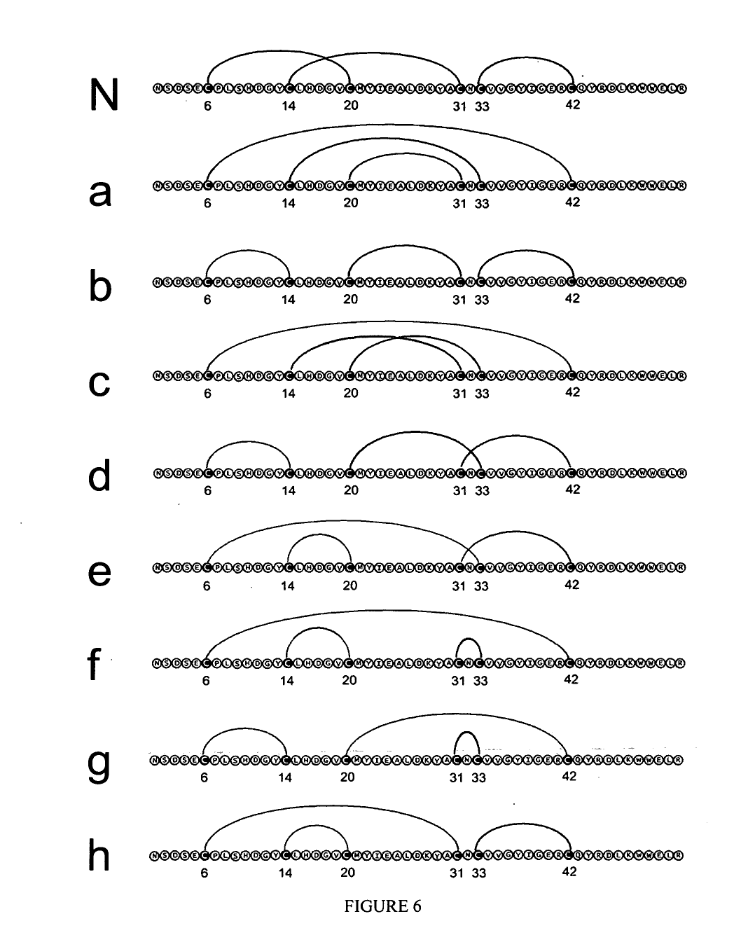

A maximum number of EGF isomers can be obtained from thermal denaturation of the native EGF. In this case, denatured EGF was shown to consist of 8 fractions of scrambled isomers (FIG. 5). This number accounts for about 60% of the 14 total possible isomers of denatured EGF. Their disulfide structures (marked from a to h) have been determined (12) (FIG. 6). The most predominant denatured isomers (“b”) adopts the bead-form disulfide pattern (1-2, 3-4, 5-6).

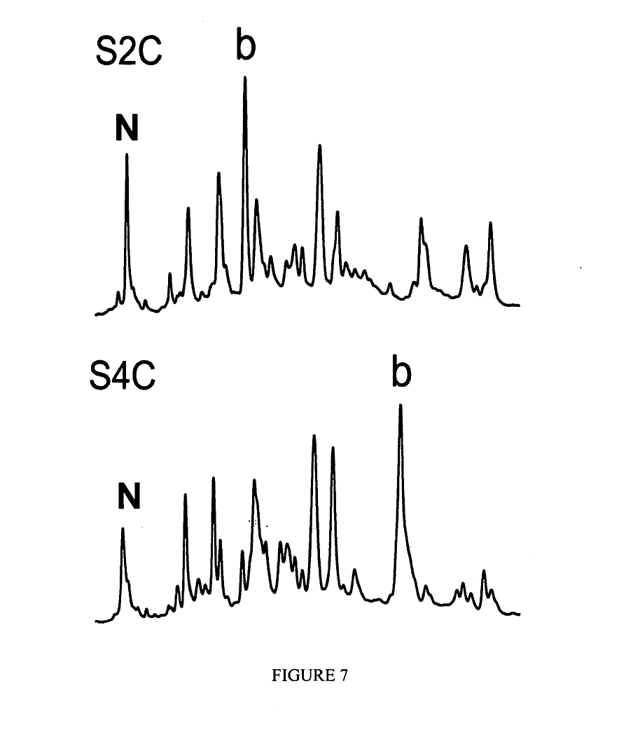

Additional isomers of EGF are generated by denaturation of two EGF mutants, EGF (S2C) and EGF (S4C), with Ser²→Cys and Ser⁴→Cys replacements respectively. Thermal denaturation of both EGF(S2C) and EGF(S4C) was shown here to produce about 24 identifiable isomers (FIG. 7).

FIG. 5 Thermal denaturation of EGF. The native EGF was dissolved in the Tris-HCl buffer (50 mM, pH 8.4) with 200 μM of Cys as a thiol catalyst. The protein concentration was 1 mg/ml. The samples were then incubated at 22° C., 37° C., 50° C. and 60° C. for 1 h, quenched by acidification and analyzed by HPLC. Denatured EGF includes 8 isomers (marked as a-h). “N” indicates the native conformation.

FIG. 6. Disulfide structures of the native EGF and eight isomers of scrambled EGF. Their structures were derived from the Edman sequencing and mass analysis of disulfide-containing peptides of thermolysin digested samples (12).

FIG. 7. Thermal denaturation of two EGF(mutants), N-EGF(S2C) and N-EGF(S4C). Denaturation was performed at 80° C. for 5 minuets in Tris-HCl buffer (0.1 M, pH 8.4) without supplementing thiol catalyst. Reactions were quenched by mixing with 2 volumes of 4% aqueous trifluoroacetic acid and analyzed by HPLC. “N” indicates the native from of EGF(mutants). “b” indicates the most predominant isomer of denatured N-EGF(mutants). About 24 fractions of-denatured isomers are identifiable in each case.

### EXAMPLE III

Production of stabilized isomers of α-synuclein. α-Synuclein is a small (14 kDa) soluble protein of unknown function and is abundant in various part of the brain. α-Synuclein is also a major component of the intracellular inclusions and abnormal neuritis (Lewy bodies and Lewy neuritis) that are characteristic of Parkinson disease (PD) (13-18). Similar to the prion protein in prion diseases and amyloid β-protein in Alzheimer's disease, several observations have shown that aggregation of α-synuclein is associated with the pathogenesis of PD and conformational change of α-synuclein represents an apparent cause leading to the process α-synuclein aggregation (13-18). Unlike the majority of native proteins which adopt defined conformations, α-Synuclein is a natively unfolded protein, exhibiting a random coil secondary structure at normal physiological conditions (19,20). Thus, the structure of α-synuclein most likely includes an assembly of conformational isomers exist in a state of equilibrium. The ability to isolate and characterize these isomers are essential to the understanding the mechanism of fibrillation of α-synuclein.

The major objective of this invention is to produce diverse and stabilized conformational isomers of human α-synuclein as potential molecules for the intervention and treatment of Parkinson diseases. Specifically, it is anticipated that some of these α-synuclein isomers will exhibit potent antigenic activity which elicit production of antibodies capable of neutralizing human α-synuclein.

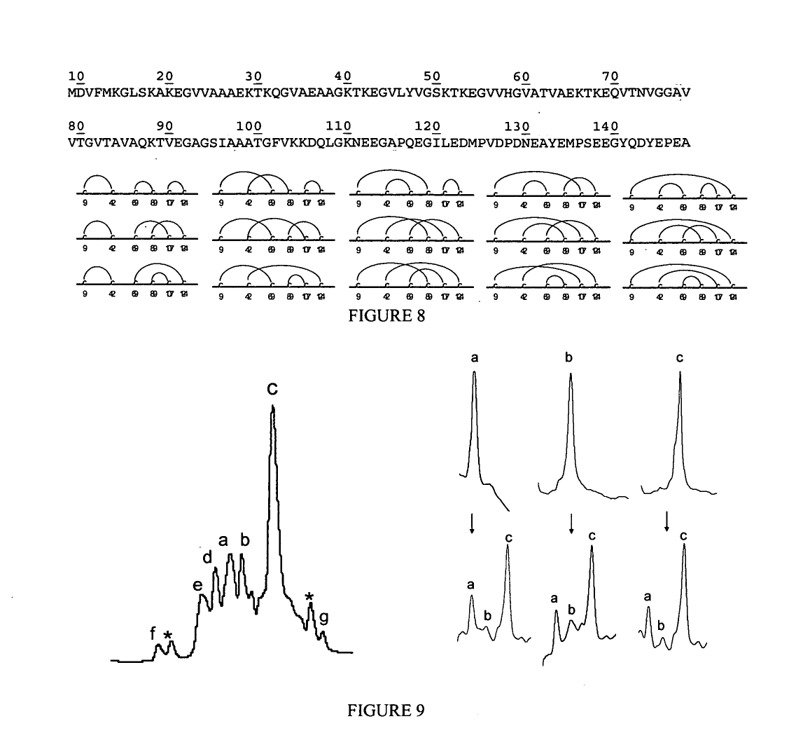

Human α-synuclein contains no cysteines. In order to prepare stabilized conformational isomers of α-synuclein, it is essential to replace Ser/Ala of the wild-type α-synuclein with Cys via site-directed mutagenesis. Mutations were created by replacing the Ser/Ala codons with Cys at six sequence positions, namely S9C, S42C, A69C, A89C, A107C, and A124C based on the constructs of the wild-type α-synuclein (FIG. 8). This allows expression and isolation of α-synuclein(6C). Oxidation of α-synuclein(6C) subsequently leads to the formation of isomers of α-synuclein(3SS). There are 15 possible isomers of α-synuclein(3SS).

The plasmid construct of human α-synuclein was obtained by PCR amplification of cDNAs that were generated by reverse transcription of total RNA isolated from SH-SY5Y using TRIzol reagent (Life Technologies, U.S.A.). The amplified products were cloned into pGEX5X-1 (Amersham Phamacia Biotech, Piscataway, N.J., U.S.A.) using XmaI and XhoI. The full length α-synuclein protein contains 140 amino acid residues. The sequence of the constructs was verified by DNA sequencing. Expression of GST-synuclein fusion proteins in BL21 [F-ompT hsdSB(rB-mB-) gal dcm] cells (Stratagene) was induced with 0.5 mM isopropyl b-D-thiogalactoside (IPTG) for 4 h at room temperature. The cultures were collected by centrifugation and the bacterial pellets were re-suspended in Cellytic B bacterial cell lysis/extraction reagent (Sigma, B-3553) containing protease inhibitor (Sigma, P8849). The GST-synuclein fusion proteins were purified from crude cell lysates under non-denaturing conditions by selective binding to glutathione-Sepharose 4B Beads (Amersham Pharmacia Biotech) following the instructions of the product. The GST-synuclein fusion proteins bound to beads were re-suspended in elution buffer (5 mM reduced glutathione in 50 mM Tris-HCl, pH 9.5) following three times' PBS washing. Then reduced glutathione was removed from samples by dialysis against Factor Xa reaction buffer (100 mM NaCl, 50 mM Tris-HCl, 1 mM CaCl₂, pH 8.0). After dialysis, the samples were incubated with Factor Xa ( 1/100, w/w, Amersham Pharmacia Biotech) at 25° C. for 16 h with gentle mixing. The cleaved proteins which already comprise heterogeneous isomers of α-synuclein(3SS) were further purified by HPLC (see FIG. 9). These purified α-synuclein(3SS) proteins were lyophilized and stored at −80° C.

FIG. 8. Ala/Ser→Cys mutations of α-synuclein sequence for the preparation of α-synuclein(6C) and the 15 potential isomers of α-synuclein(3SS) that may be produced from α-synuclein(6C) via disulfide oxidation and scrambling. α-Synuclein sequence contains no cysteine. α-Synuclein(6C) was produced via Ala/Ser→Cys site-directed mutations at Ser⁹, Ser⁴², Ala⁶⁹, Ala⁸⁹, Ala¹⁰⁷, and Ala¹²⁴.

Factor Xa cleavage of GST-synuclein fusion proteins were separated and isolated by HPLC using the conditions described in the legend of FIG. 9. The results show that under non-denaturing conditions, α-synuclein(3SS) includes heterogeneous conformational isomers. At least 7 fractions of α-synuclein(3SS) isomers (marked by lower case from a-g in FIG. 9) were identified by this method.

All 7 isomers of α-synuclein(3SS) were purified, modified with vinylpyridine before and after DTT reduction, and analyzed by MALDI mass spectrometry. The results verify that all 7 α-synuclein (3SS isomers contains 3 disulfide bonds and no free cysteines).

FIG. 9. (Left) HPLC separation of isomers of α-synuclein(3SS). Seven fractions of α-synuclein(3SS) isomers were purified by HPLC using the following conditions. Column was Zorbax 300XB-C18, 250 mm×4.6 mm 5 μm. Buffer A was 0.1% TFA in water. Buffer B was 0.086% TFA in acetonitrile/water (9:1, by volume). The gradient of elution was 20% B to 70% B linear in 30 min. The flow rate was 0.5 ml/min. Column temperature was 23° C. (Right) Demonstration that isomers of α-synuclein(3SS) exist in equilibrium under non-denaturing conditions. Purified isomers “a”, “b” and “c” of α-synuclein(3SS) were each re-constituted in the Tris-HCl buffer (0.1 M, pH 8.4) containing 0.2 mM 2-mercaptoethanol. They were allowed to incubate at 22° C. for 4 hours, quenched with 4% trifluoroacetic acid and analyzed by HPLC.

### EXAMPLE IV

Production of stabilized isomers of Prion protein. The prion diseases, including the mad-cow disease and the human version of Creutzfeldt-Jacob disease, are caused and transmitted by the infectious scrapie prion (PrP^{SC}). PrP^{SC} is an isomer of the benign cellular prion (PrP^{C}) (21-23). Although physiological conditions which induce PrP^{C}.→PrP^{SC} conversion has yet to be identified, the course of this conformational change is characterized by a decrease of α-helical structure, an increase of β-sheet content and the formation of PrP^{SC} amyloid (24). The molecular basis of this conformational change is central to our understanding and intervention of prion diseases. Despite mounting efforts, to date, two aspects of the crucial data concerning the prion diseases remain to be elucidated: (a) The detailed structure of the scrapie prion (PrP^{SC}) and its strain related isoforms; and (b) The pathway and mechanism for the conversion of the cellular prion (PrP^{C}) to the scrapie prion (PrP^{SC}) (24).

The main objective of this invention is to produce diverse and stabilized conformational isomers of mouse prion protein as candidates for the intervention and treatment of prion diseases. Specifically, it is expected that some of these prion isomers will be potent antigens which elicit production of antibodies capable of neutralizing either PrP^{C} or PrP^{SC}.

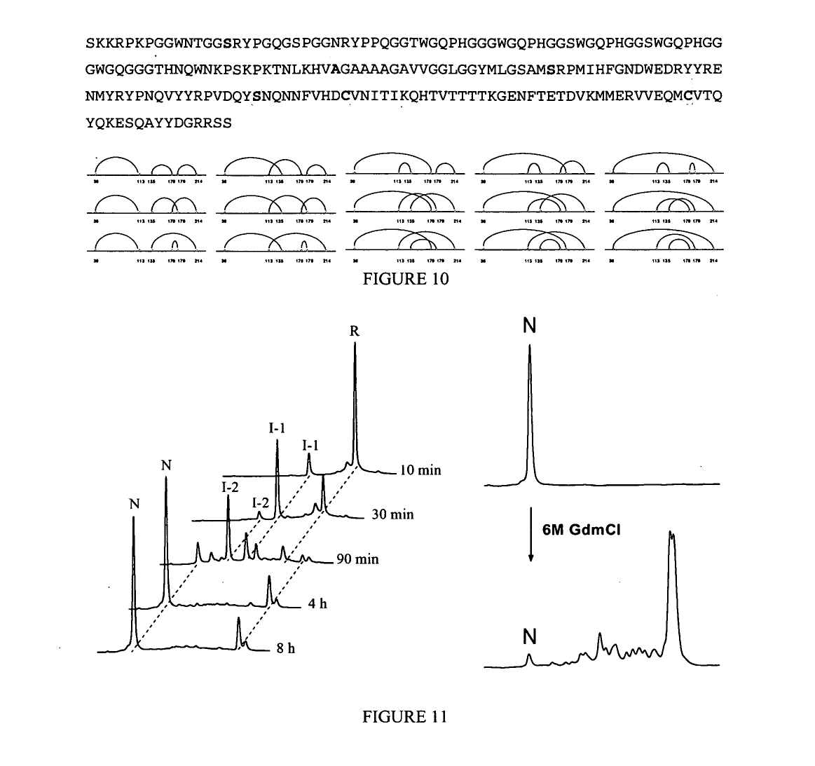

The prion protein contains only one disulfide bond Cys¹⁷⁹-Cys²¹⁴. In order to generate diverse isomers of prion protein, it is necessary to introduce 4 additional Cys. One Ala residue at position 113 and three Ser residues at positions 36, 135 and 170 were replaced by Cys using the plasmid that encode the mouse prion protein mPrP(23-231) (FIG. 10). This allows the mutated mPrP923-231(6C), under oxidative and selected denaturing conditions to adopt 15 possible 3-disulfide isomers (FIG. 10).

Plasmid pRBI-PDI-T7 harboring the mPrp(23-23 1) cDNA was mutated via a QuickChange® Site-Directed Mutagenesis kit. The obtained plasmid with the right mutations was transformed into cells of E.Coli BL21(DE3) and grown at 37° C. Cells were grown and mutated protein was isolated according to the methods described in (25). The fully reduced mPrp(23-231)(6C) isolated form the inclusion body was reconstituted in the Tris-HCl buffer (pH 8.7) containing 3.5 M urea and 1 μM CuSO4, and allowed to refold for 16hours at 23° C. The process of folding was monitored by HPLC analysis (FIG. 11). The refolded product designated as N-mPrP(23-231)(6C) was shown to include 3 disulfide bonds as evaluated by MALDI mass spectrometry following vinylpyridine modification.

For the preparation of prion protein isomers, N-mPrP(23-231)(6C) was dissolved in the alkaline buffer (20-200 mM, pH 7.0-8.5) containing 0.05-0.4 mM of thiol catalyst (e.g., 2-mercaptoethanol) and selected denaturing conditions (GdmCl, GdmSCN, organic solvents, elevated temperature etc.). The reaction was allowed to reach equilibrium and was typically performed at 23° C. for 24 hours. Thiol agents other than 2-mercaptoethanol may also be used as thiol catalyst. The denatured sample was subsequently acidified with an equal volume of 4% trifluoroacetic acid and stored at −20° C. For large scale production, denaturant and thiol agent were removed by gel filtration (e.g. PD-10 or NAP-5 columns from Pharmacia AG), eluted with 1% trifluoroacetic acid. Denatured scrambled isomers are totally stable at −20° C. for at least one year, which may be fractionated and isolated by reversed phase HPLC.

FIG. 10. Ala/Ser→Cys mutations of mPrP sequence for the preparation of mPrP(6C) and the 15 potential isomers of mPrP(3SS) that may be generated from disulfide oxidation and scrambling of mPrP(6C). Native mPrP already contains one disulfide bonds bridged by Cys179 and Cys214. Three Ser residues at positions 36, 135, 170 and one Ala residue at position 113 were replacedby Cys.

Oxidative folding of the fully reduced mPrP(6C) generates a single predominant product, designated as N-mPrP(3SS)(indicated by N at the left panel of FIG. 11). A systematic study has shown that an optimized condition to generate N-mPrP(3SS) is in the Tris-HCl buffer (0.1 M, pH 8.4) containing 3.5M urea and 1 μM CuSO₄, a condition similar to the one that promote efficient oxidative folding of wild-type mPrP. Urea is required for solubilization of the mutant prion protein. Mass analysis of mPrP(3SS) before and after reduction (and vinylpyridine modification) revealed that N-mPrP(3SS) contains 3-disulfide bonds and no free cysteine.

Isomers of prion protein can be produced by GdmCl denaturation of N-mPrP(3SS) in the presence of 2-mercaptoethanol. Under these conditions, denatured N-mPrP(3SS) includes 2 major and 10 minor fractions of mPrP(3SS) isomers. Mass analysis after vinylpyridine modification confirms that these 12 fractions of isomers all contain 3-disulfide bonds.

FIG. 11. (Left) Oxidative folding of reduced mPrP(6C). Folding of reduced mPrP(6C) was performed in the Tris-HCl buffer (0.1 M, pH 8.4) containing 3.5M urea and 1 PM CuSO₄. Aliquots of samples were removed at different time points and analyzed by HPLC using the following conditions. Column was Zorbax 300A C18, 5 mm, 4.6×250 mm. Buffer A was 0.088% TFA in water, and buffer B was 0.084% TFA in 90% acetonitrile and 10% water. The gradient was 28% to 52% B linear in 30 min. The flow rate of 0.5 ml/min. R indicates the fully reduced mPrP(6C) with 6 free cysteines. N stands for fully oxidized N-mPrP(3SS) with 3 disulfide bonds. (Right) Production of isomers of prion protein via disulfide scrambling of N-mPrP(3SS). N-mPrP(3SS) (indicated by N) isolated from the oxidative folding was reconstituted in the Tris-HCl buffer (0.1 M, pH 8.4) containing 6M GdmCl and 0.1 mM 2-mercaptoethanol. The denaturation reaction was allowed at 22° C. for 16 hours, quenched with 4% trifluoroacetic acid and analyzed by HPLC. Two major and 10 minor fractions of mPrP(3SS) isomers are identifiable.

### EXAMPLE V

Production of stabilized isomers of Amyloid β-protein (Aβ). β-Amyloid protein (Aβ) is a small hydrophobic peptide occurs in two principal lengths, Aβ40 and Aβ42. They are generated from Aβ precursor (APP) in vivo via proteolytic processing by two aspartyl proteases, namely β- and α-secretases (26-28). Aggregation of Aβ to from oligomeric and polymeric (fibrillar) assemblies represents a major chemical event in the development of Alzheimer's diseases. They form senile (neuritic) plaques which have been shown to degenerate axons and neurites within and surrounding the amyloid deposits. It is now generally believed that formation of toxic Aβ plaques is associated with the progression of AD (29,30).

Thus, Aβ represents a rational target for developing the treatment of AD. Currently, this has been actively pursued in two different option (29). First, to reduce the in vivo production of Aβ through inhibition of β- and α-secretases required for the processing of APP. Second, to clear Aβ from the brain through immunological approach. Either by the strategy of active immunization using Aβ42 or by the strategy of passive immunization using mAb raised against Aβ.

The methods of the present invention have been used to produce diverse and stabilized conformational isomers of human Aβ as candidates for the intervention and treatment of Alzheimer's diseases. Specifically, some of these Aβ isomers are likely to exhibit potent antigenic activity that elicits production of antibodies capable of neutralizing monomeric Aβ as well as oligomeric and polymeric Aβ.

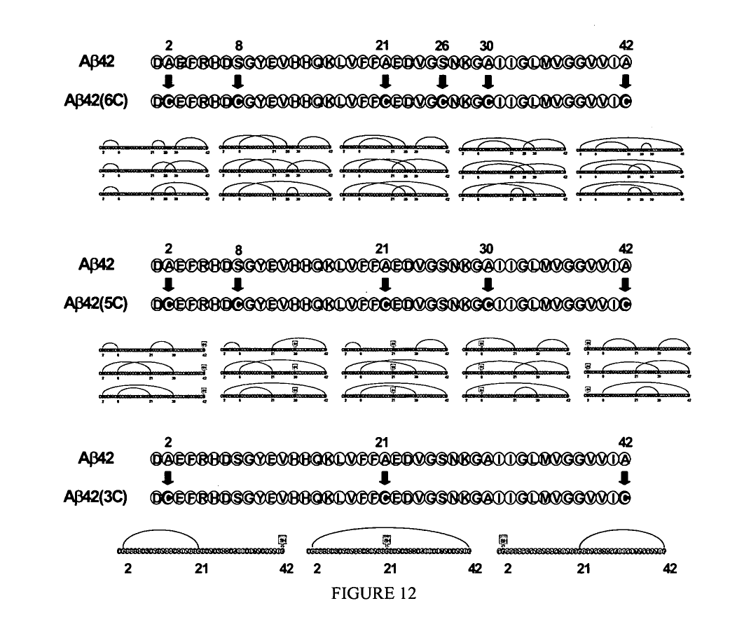

Human Aβ contains no cysteines. In order to prepare stabilized conformational isomers of Aβ, it is essential to replace Ser/Ala of the wild-type Aβ with Cys. Aβ42 includes six Ser/Ala at positions 2, 8, 21, 26, 30 and 42. They will be entirely or partially replaced by Cys to generate three mutants Aβ42(6C), Aβ42(5C) and Aβ42(3C) (FIG. 12). These peptides will be prepared by chemical synthesis.

Fully reduced Aβ42(6C), AB42(5C) and Aβ42(3C) will be allowed to refold via oxidative folding in the Tris-HCl buffer (pH 7.5-8.5) in the presence and absence of 2-mercaptoethanol (0.1-0.25 mM). Further folding studies may be conducted systematically under conditions that including (a) varying concentrations of urea (1-8M), GdmCl (1-8M) and GdmSCN (1-6M); (b) varying concentrations of organic solvent (1-10M); and (c) varying temperature (20-80° C.). Folded (oxidized) isomers of Aβ42(6C) will be quenched by acidification. Folded (oxidized) isomers of Aβ42(5C) and Aβ42(3C) will be treated with thiol specific reagents to block the free Cys. All folded isomers are subsequently separated and isolated by HPLC.

Isomer selection. Oxidative folding of Aβ42(6C) and Aβ42(5C) may each generate 15 potential disulfide isomers. Oxidative folding of Aβ42(3C) may produce 3 disulfide isomers. Their disulfide structures are presented in FIG. 12.

FIG. 12. Ala/Ser→cCys replacements of Aβ42 for the synthesis of Aβ42(6C), Aβ42(5C) and Aβ42(3C), and potential isomers that may be generated. Disulfide scrambling of Aβ42(6C) and Aβ42(5C) may each produced 15 different isomers. Disulfide scrambling of Aβ42(3C) can potentially generate only 3 disulfide isomers. The odd number of Cys in isomers of Aβ42(5C) and Aβ42(3C) will be blocked by chemical reagents, such as iodoacetamide or vinylpyridine.

### EXAMPLE VI

Immunogenicity of non-native isomers of α-lactalbumin. In the section 3 (example I), it has been demonstrated that preferred populations of conformational isomers of α-lactalbumin can be produced using selected denaturing conditions. Some of the isomers are partially unfolded (e.g. isomers b & c) and some of them are more extensively unfolded (e.g. isomers a & b). These diverse isomers are expected to exhibit different structural and biological properties, including their ability to elicit immune response.

The major objective here is to demonstrate that isomers of α-lactalbumin may display distinct antigenic activity. Specifically, the aim is to show that: (a) denatured isomers of α-lactalbumin may be more immunogenic than the native α-lactalbumin; and (b) antibodies raised against denatured isomers of α-lactalbumin may recognize and neutralize the native α-lactalbumin.

Groups of normal Balb/c mice (5/group) were immunized with the native α-lactalbumin (column A in table 1) and three different stabilized denatured isomers of α-lactalbumin, namely isomers “a”, “c” and “d” (columns B, C and D in table 1) as antigens in CFA and than boosted twice with the same antigen in IFA. Individual mouse was bled and the antibody titers in each mouse were determined by ELISA using plates pre-coated with A, B, C or D antigen (rows).

For the disulfide structures of these four isomers of α-lactalbumin, please see FIG. 4. The results show that: (a) the native protein is not very immunogenic at all, as expected. Only one mouse produced significant antibody against all four antigens (see red readings). One additional mouse produced antibody against isomer “a”. (b) Isomer “a” (Column B) is very immunogenic and all immunized mice produced antibody against all four antigens, albeit at different levels as expected; (c) Isomers “c” and “d” are non-immunogenic (columns C and D).

These data demonstrate the use of denatured and stabilized conformational isomers of the otherwise non-immunogenic proteins as immunogenic vaccines and for use in epitope selection.

TABLE 1
Detected (O.D.)

| Immunized | Animal | | | | |
|---|---|---|---|---|---|
| with | No. | A | B | C | D |
| A | 1 | 0.062 | 0.053 | 0.047 | 0.061 |
| | 2 | 0.058 | 0.361 | 0.054 | 0.056 |
| | 3 | 0.057 | 0.045 | 0.042 | 0.052 |
| | 4 | 1.331 | 1.268 | 1.157 | 1.308 |
| | 5 | 0.039 | 0.039 | 0.04 | 0.04 |
| B | 1 | 1.049 | 0.396 | 0.122 | 0.094 |
| | 2 | 1.322 | 0.77 | 0.316 | 0.122 |
| | 3 | 1.361 | 1.276 | 0.620 | 0.270 |
| | 4 | 0.995 | 0.223 | 0.276 | 0.203 |
| | 5 | 1.142 | 0.938 | 0.632 | 0.191 |
| C | 1 | 0.084 | 0.072 | 0.061 | 0.076 |
| | 2 | 0.064 | 0.075 | 0.060 | 0.067 |
| | 3 | 0.051 | 0.052 | 0.051 | 0.051 |
| | 4 | 0.040 | 0.039 | 0.046 | 0.041 |
| | 5 | 0.045 | 0.047 | 0.048 | 0.046 |
| D | 1 | 0.057 | 0.044 | 0.048 | 0.053 |
| | 2 | 0.042 | 0.042 | 0.051 | 0.047 |
| | 3 | 0.044 | 0.046 | 0.05 | 0.045 |
| | 4 | 0.037 | 0.052 | 0.053 | 0.055 |
| | 5 | 0.104 | 0.038 | 0.041 | 0.039 |

| | | | | |
|---|---|---|---|---|
| Normal | 0.06 | 0.06 | 0.056 | 0.050 |
| Blank | 0.03 | 0.03 | 0.03 | 0.034 |

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

(1) Blundell, T. L. (1996) Nature 384, 23-26.

(2) Dill, K. A., and Shortle, D. (1991) Annu. Rev. Biochem. 60, 795-825.

(3) Tanford, C. (1968) Adv. Protein Chem. 23, 121-282.

(4) Chang, J.-Y. (1999) J. Biol. Chem. 274, 123-128.

(5) Chang, J.-Y., and Li, L. (2001) J. Biol. Chem. 276, 9705-9712.

(6) Kuwajima, K. (1989) Proteins Struct. Funct. Genet. 6, 87-103.

(7) Barrick, D., and Baldwin, R. L. (1993) Protein Sci. 2, 869-876.

(8) Schlessinger, J., and Ullrich, A. (1992) Neuron 9, 383-391.

(9) Montelione, G. T., Wuthrich, K., Burgess, A. W., Nice, E. C., Wagner, G., Gibson, K. D. and Scheraga, H. A. (1992) Biochemistry 31, 236-249.

(10) Doolittle, R. F., Feng, D. F., and Johnson, M. S. (1984) Nature 307, 558-560.

(11) Campbell, I. D., and Bork, P. (1993) Curr. Opin. Struct. Biol. 3, 385-392.

(12) Chang, J-Y and Li. L. (2002) J. Protein. Chem, 21, 203-213.

(13) Hashimoto M, Rockenstein E, Crews L, Masliah E. (2003) 9: Neuromolecular Med. 4, 21-36.

(14) Klucken J, Shin Y, Masliah E, Hyman B T, McLean P J. (2004) J Biol Chem. 279, 25497-25502.

(15) Zhu M, Fink A L. (2003) J Biol Chem. 278, 16873-16877.

(16) Kessler J C, Rochet J C, Lansbury P T Jr. (2003) Biochemistry. 42, 672-678.

(17) Biere A L, Wood S J, Wypych J, Steavenson S, Jiang Y, Anafi D, Jacobsen F W, Jarosinski M A, Wu G M, Louis J C, Martin F, Narhi L O, Citron M. (2000) J Biol Chem. 275, 34574-34579.

(18) Conway K A, Harper J D, Lansbury P T. (1998). Nat Med. 4, 1318-1320.

(19) Munishkina L A, Henriques J, Uversky V N, Fink A L. (2004) Biochemistry. 43, 3289-3300.

(20) Maiti N C, Apetri M M, Zagorski M G, Carey P R, Anderson V E. (2004) J Am Chem Soc. 126, 2399-2408.

(21) Pruisner, S. B. (1997) Prion disease and BSE crisis. Science 278, 245-251.

(22) Pruisner, S. B. (1999) Prions. Proc. Natl. Acad. Sci. U.S.A. 95, 13363-13383.

(23) Horiuchi, M., and Caughey, B. (1999) Structure Fold. Des. 7, R231-240.

(24) Cohen, F. E., and Prusiner, S. B. (1998) Annu. Rev. Biochem. 67, 793-819.

(25) Lu, B. Y., Beck, P. J., and Chang, J.-Y. (2001) Eur. J. Biochem. 268, 3767-3773.

(26) Shoji, M., Golde, T. E., Ghiso, J., Cheung, T. T., Estus, S., Shaffer, L. M., Cai, X. D., McKay, D. M., Tintner, R., Frangione, B., et al. (1992) Science 258, 126-129.

(27) Esch, F. S., Keim, P. S., Beattie, E. C., Blacher, R. W., Culwell, A. R., Oltersdorf, T., McClure, D., Ward, P. J. (1990) Science 248, 1122-1124.

(28) Sisodia, S. S., Koo, E. H., Beyreuther, K., Unterbeck, A., and Price, D. L. (1990) Science. 248, 492-495.

(29) Selkoe, J. D., and Schenk, D. (2003) Annu. Rev. Pharmacol. Toxicol. 43, 545-584.

(30) Hardy, J. (1997) TIBS 20, 154-159.

What is claimed is:

1. A vaccine comprising one or more isolated and purified non-native, stabilized conformational protein isomer antigens.

2. The vaccine of claim 1, wherein the protein isomer comprises three or more cysteines.

3. The vaccine of claim 1, wherein the protein isomer comprises a wild-type protein that has been modified to comprise three or more cysteines.

4. The vaccine of claim 1, wherein one or more residues selected from the group consisting essentially of Alanine, Serine and Threonine are modified to one or more cysteines.

5. The vaccine of claim 1, wherein the vaccine comprises one or more pools of protein isomers.

6. The vaccine of claim 1, further comprising one or more adjuvants.

7. The vaccine of claim 1, wherein the vaccine is adapted for intramuscular, intravenous, subcutaneous, pulmonary, oral, ocular, topical, sublingual, intraperitoneal, intraosseal, rectal, vaginal or intranasal injection.

8. The vaccine of claim 1, wherein the isomer is derived from prions, α-synuclein, amyloid β-protein, CD4 or gp120.

9. The vaccine of claim 1, wherein the isomer is associated with a conformational disease selected from the group consisting of prion-associated diseases, mad cow disease, scrapie, Creutzfeldt-Jacob disease, familial insomnia, Alzheimer disease, Parkinson disease, α₁-antitrypsin deficiency and cystic fibrosis.

10. The vaccine of claim 1, wherein the one or more protein isomers comprise protein aggregates.

11. A non-native, protein isomer library comprising one or more non-native, stabilized conformational protein isomers antigens.

12. The library of claim 11, wherein the library comprises one or more pools of pools of non-native, stabilized protein isomers.

13. A method for producing non-native and stabilized protein isomer antigens comprising contacting a protein antigen comprising three or more sulfydryl side-groups in the presence of varying denaturing conditions against one or more concentrations of a thiol agent.

14. The method of claim 13, wherein the thiol agent is selected from 2-mercaptoethanol, reduced glutathione, cysteine, dithiothreitol, and thiol-containing chemicals.

15. The method of claim 13, the denaturing conditions include one or more concentrations of Urea, guanidinium chloride, guanidinium isothiocyanate, organic solvents, and mixture or combinations thereof.

16. The method of claim 13, wherein the denaturing condition is selected from the group consisting of urea, guanidine hydrochloride, guanidinium isothiocyanate, organic solvents, elevated temperature, extreme pH, surfactants and detergents, and mechanical forces such as shaking, shearing, ultrasound, radiation and pressure.

17. The method of claim 13, further comprising exposing the protein to temperatures ranging from between about 0 and 90 degrees centigrade.

18. A method for producing a non-native, stabilized protein isomer antigen comprising contacting a protein antigen comprising two or more sulfydryl groups in the presence of varying denaturing conditions against one or more concentrations of a thiol agent.

19. A method for isolating a stable, non-native conformational protein isomer comprising the steps of:
contacting a protein in a titration array comprising one or more wells comprising one or more concentrations of a protein denaturing agent and one or more concentrations of a reducing agent; and
vaccinating an animal with the one or more a stable, non-native conformational protein isomers formed in the one or more cells.

20. The method of claim 19, wherein the one or more stable, non-native conformational protein isomer from different wells are compared for immunogenicity.

21. The method of claim 19, wherein the one or more stable, non-native conformational protein isomer from different wells are compared for cross-reactivity.

22. The method of claim 19, wherein the reducing agent is selected from 2-mercaptoethanol, reduced glutathione, cysteine, dithiothreitol, and thiol-containing chemicals.

23. The method of claim 19, the protein denaturing agent includes one or more concentrations of Urea, guanidinium chloride, guanidinium isothiocyanate, organic solvents, and mixture or combinations thereof.

24. A vaccine comprising one or more non-native, immunogenic proteins comprising an Aβ protein comprising six cysteine residues at positions 2, 8, 21, 26, 30 and 42.

25. A vaccine comprising one or more non-native, immunogenic proteins comprising an prion protein comprising four or more cysteine residues at positions 36, 113, 135 and 170.
